(19) United States
(12) Patent Application Publication
(10) Pub. No.: US 2016/0029904 A1
(43) Pub. Date: Feb. 4, 2016

(54) AUTOMATED BLOOD PRESSURE MEASUREMENT SYSTEM

(71) Applicant: Welch Allyn, Inc., Skaneateles Falls, NY (US)

(72) Inventor: David E. Quinn, Auburn, NY (US)

(21) Appl. No.: 14/450,510

(22) Filed: Aug. 4, 2014

(51) Int. Cl. A61B 5/0225; A61B 5/00; A61B 5/02; A61B 5/022

(52) U.S. Cl. CPC A61B 5/0225; A61B 5/02233; A61B 5/742; A61B 5/7278; A61B 5/7282; A61B 5/02028; A61B 5/0024; A61B 5/486

(57) ABSTRACT

A non-invasive blood pressure measurement system is disclosed. The system can include a processing unit, a non-invasive blood pressure (NIBP) sensor, a first altitude sensor and a second altitude sensor. The NIBP sensor can be configured to transmit a blood pressure signal to the processing unit. The first and second altitude sensors can be configured to generate and transmit first and second height measurements, respectively. The processing unit can be configured to generate an adjusted blood pressure measurement using the first height measurement, which can be the height of a patient's heart, and the second height measurement, which can be the height of the NIBP sensor. Methods for determining the blood pressure of a patient using the system are also disclosed.

### BACKGROUND

Non-invasive blood pressure measurements require the measuring device to be at heart level because hydrostatic pressure in the arterial system can otherwise cause an incorrect measurement. Hydrostatic pressure can cause a significant impact to such a measurement, in some cases equating to a 2 mm Hg per inch change when the blood pressure measuring device is above or below the heart.

Some people with blood pressure conditions have a blood pressure measurement device at their own home as a means for monitoring their blood pressure more frequently than their visits to a doctor's office. Without proper guidance, or adherence to that guidance, there is a risk that the blood pressure measurement device could be incorrectly placed during a reading, resulting in an incorrect measurement.

Further, orthostatic hypotension is a slow or absent response of the body's blood pressure regulation to a change in vertical position. The condition can cause dizziness, lightheadedness or fainting if a person with orthostatic hypotension stands up too quickly. Traditional methods for screening for orthostatic hypotension include measuring blood pressure of a patient sitting and measuring the blood pressure of that patient after the patient stands up.

### SUMMARY

In one aspect, a non-invasive blood pressure measurement system is disclosed. The non-invasive blood pressure measurement system can include a processing unit, a non-invasive blood pressure sensor configured to transmit a blood pressure signal to the processing unit, a first altitude sensor configured to generate a first height measurement and transmit the first height measurement to the processing unit, and a second altitude sensor configured to generate a second height measurement and transmit the first height measurement to the processing unit. The processing unit can be configured to generate an adjusted blood pressure measurement using the first height measurement, the second height measurement and the blood pressure signal.

In embodiments, the non-invasive blood pressure sensor is a cuff including a pressure interface opening, and wherein the first altitude sensor is positioned at the cuff and the processing unit can be configured to calculate the adjusted blood pressure measurement after receiving both the first and the second height measurements. In embodiments, the system can further include a display module that displays the calculated systolic and diastolic blood pressure. One or both of the first and the second altitude sensors can be configured to transmit the height measurement to the processing unit wirelessly. The blood pressure signal can include the unadjusted systolic and diastolic blood pressure. The blood pressure sensor can be configured to transmit the blood pressure signal to the processing unit wirelessly and the second altitude sensor can be positioned at the heart level of a patient.

In another aspect, a method for determining the blood pressure of a patient is disclosed. In embodiments, the method can include receiving a blood pressure measurement from a blood pressure sensor, receiving a first height measurement from a first altimeter, receiving a first height measurement from a second altimeter, adjusting the blood pressure measurement using the first height measurement from the first altimeter and the first height measurement from the second altimeter, producing an adjusted blood pressure measurement comprising the systolic and diastolic pressure, and displaying the adjusted blood pressure measurement. In some embodiments the method can also include positioning the first altimeter at the blood pressure sensor and positioning the second altimeter at a heart of the patient. The patient can be a person or an animal.

In embodiments, the blood pressure measurement can be received from a pressure transducer and each of the blood pressure measurement, the first height measurement and the second height measurement can be received wirelessly. The example method can also include receiving a second height measurement from the first altimeter, initiating a second blood pressure measurement, receiving a second height measurement from the second altimeter, and adjusting the second blood pressure measurement using the second height measurement from the first altimeter and the second height measurement from the second altimeter, producing a second adjusted blood pressure measurement comprising the systolic and diastolic pressure. The measurements can be recorded at different patient positions, such as, for example, laying down, sitting, and standing.

The adjusted blood pressure measurement and the second adjusted blood pressure measurement can be used to diagnose orthostatic hypotension. The blood pressure sensor can be a cuff including a pressure interface opening. The second altimeter can be positioned at the heart level of the patient.

In yet another aspect, a tangible, non-transitory computer readable medium containing computer executable instructions which, when executed by a computer, perform a method for determining a corrected blood pressure measurement is disclosed. The method can include the steps of receiving a blood pressure measurement from a blood pressure sensor, receiving a first height measurement from a first altimeter, receiving a first height measurement from a second altimeter, adjusting the blood pressure measurement using the first height measurement from the first altimeter and the first height measurement from the second altimeter, producing an adjusted blood pressure measurement comprising the systolic and diastolic pressure, and displaying the adjusted blood pressure measurement.

### DESCRIPTION OF THE FIGURES

FIG. 1 illustrates a block diagram of an example blood pressure measurement system.

FIG. 2 illustrates an example wireless blood pressure measurement system.

FIG. 3 illustrates an example blood pressure cuff with an altimeter.

FIG. 4 illustrates a second example blood pressure cuff with an altimeter.

FIG. 5 illustrates example locations for altimeters on a human body.

FIG. 6 is a flow chart illustrating an example method for using at least two altimeters in conjunction with a blood pressure sensor.

FIG. 7 is a flow chart illustrating an example method for using an altimeter-blood pressure sensor system.

FIG. 8 is a flow chart illustrating an example method of conducting an orthostatic blood pressure test.

FIG. 9 is a flow chart illustrating a processor-implemented method of monitoring two or more altimeters.

FIG. 10 is a flow chart illustrating a processing unit's interaction with a mobile blood pressure sensor.

FIG. 11 illustrates example locations on a human body for wearing a wearable blood pressure sensor including an altimeter.

FIG. 12 is a block diagram illustrating physical components of a computing device with which examples and embodiments of the disclosure can be practiced.

### DETAILED DESCRIPTION

Blood pressure measurements are sensitive to the location of the pressure measurement site because of the weight of blood. In humans, the hydrostatic pressure effect can be approximately 2 mm Hg per inch below the approximate level of the heart. For example, a blood pressure measurement of 120/80 at heart level, for the same person, can change to be 130/90 if the measurement site is about 5 inches below the heart level. Common human blood pressure measurement sites include locations that are not at heart level, such as on a forearm, thigh, ankle, wrist, or arm of a person lying on their side. Thus, without correction for a measurement site that is not level with the heart, a normal blood pressure could be incorrectly determined as hypertensive, and vice versa. The instant disclosure is generally directed towards accounting for the difference between the height of the blood pressure measurement site and the location of the heart.

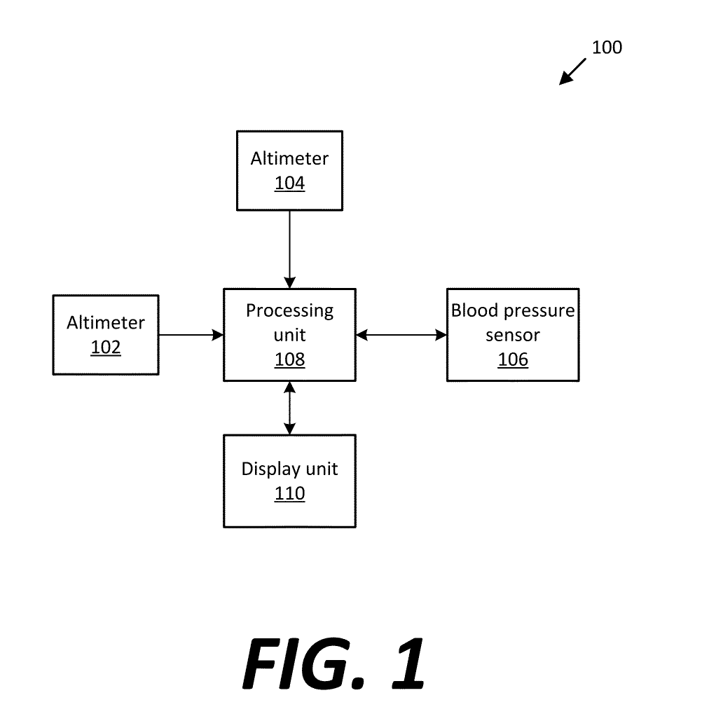

FIG. 1 is a block diagram illustrating the components of an example blood pressure measurement system 100. The example system 100 includes a first altimeter 102, a second altimeter 104, a blood pressure sensor 106, a processing unit 108 and a display unit 110. Other embodiments of the system can include more or fewer components. In embodiments, the altimeters 102 and 104 are in communication with the processing unit 108 via one or more wired or wireless protocols, such as Bluetooth, Wi-Fi, Zigbee, etc.

In some embodiments, the altimeters 102 and 104 are configured to measure and transmit altitude measurements. In some embodiments, the altimeters 102 and 104 are configured to communicate with each other and determine the vertical distance between the two altimeters 102 and 104. As used herein, “vertical distance” means the distance between the two sensors as measured in the direction of gravity.

The altimeters 102 and 104 can be micro altitude sensors. For example, the altitude sensors 102 and 104 can be an MS5607-02BA03 sensor from Measurement Specialties (Hampton, Va.), an LPS331AP sensor from STMicroelectronics (Geneva, Switzerland), or an FKS-111 air pressure sensor from Fuji & Co. (Osaka, Japan).

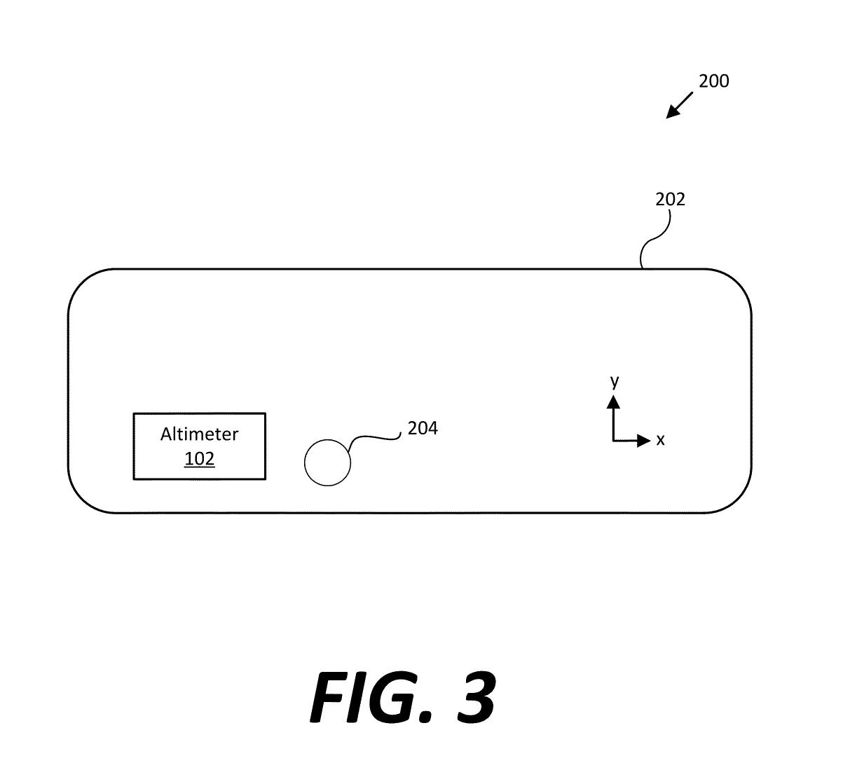

In embodiments, the processing unit 108 is configured to receive an altitude measurement from the first 102 and second 104 altimeters. One of the altimeters can be placed on or near the blood pressure measurement site and the other altimeter can be placed at heart level. Potential arrangements of the altimeters 102 and 104 and the blood pressure sensor 106 are shown and described in more detail below with reference to FIGS. 3-5 and 10.

The processing unit 108 can also be configured to communicate with a blood pressure sensor 106 to, for example, initiate a blood pressure measurement and/or receive one or more blood pressure measurements. In some embodiments, the processing unit 108 is configured to receive an analog input from the blood pressure sensor 106, such as air pressure pulses, and convert that air pressure signal to a blood pressure measurement for display on a display unit 110. Example components that can comprise the processing unit 108 are shown and described in more detail with reference to FIG.

In embodiments, the display unit 110 is configured to receive a measurement or calculation of a patient's blood pressure and display the systolic and diastolic blood pressure measurements. The display unit 110 can be configured to display other vital signs of the patient, including, for example, heart rate, respiration rate, temperature, SpO₂ (saturated oxygen), mean arterial pressure (MAP), and ETCO₂ (end-tidal carbon dioxide).

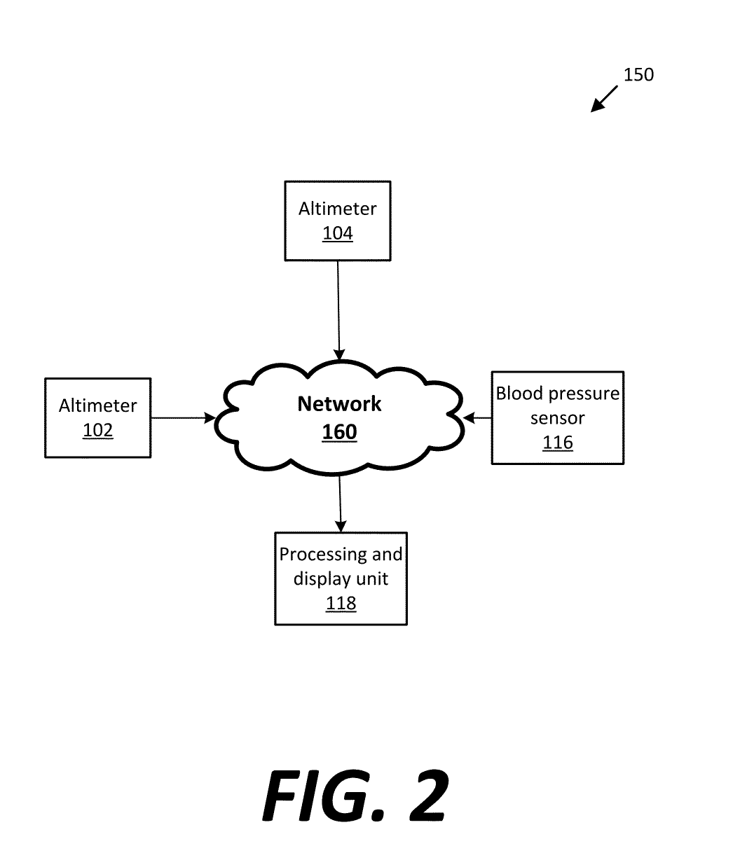

FIG. 2 is a block diagram illustrating the components of an example wireless blood pressure measurement system 150. The example system 150 includes a first altimeter 102, a second altimeter 104, a blood pressure sensor 116, a processing and display unit 118, and a network 160. Other embodiments of the system can include more or fewer components. In embodiments, the altimeters 102 and 104 are in communication with the processing and display unit 118 via the wireless network 160. In embodiments, the blood pressure sensor 116 is in communication with the processing and display unit 118 via the wireless network 160.

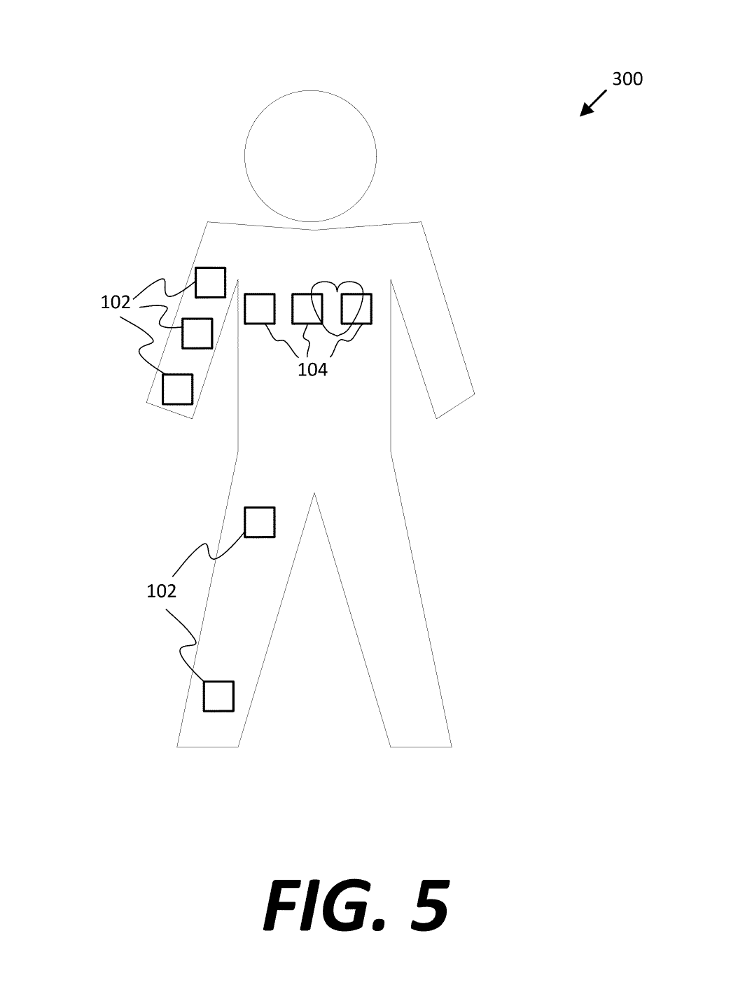

The example blood pressure sensor 116 measures the blood pressure of a person and wirelessly transmits one or more measurements to the processing and display unit 118 via the network 160. The example blood pressure sensor 116 includes a wireless communication component for sending and/or receiving data to or from the processing and display unit 118 via the network 160. In embodiments, the blood pressure sensor 116 can be placed on, for example, an arm, wrist, finger, etc. Additional locations are shown and described in more detail with reference to FIG. 5, below.

The example processing and display unit 118 receives readings from the two altimeters 102 and 104 as well as the blood pressure measurement reading from the blood pressure sensor 116. In embodiments, the processing and display unit 118 can adjust the measurement received from sensor 116 based on the altimeter readings 102 and 104 using one or more modification algorithms. The processing and display unit 118 can then display the systolic and diastolic measurements as well as the mean arterial pressure.

In some embodiments, the processing and display unit 118 are in the same vicinity as the altimeters 102 and 104 and blood pressure sensor 116, such as a person using the sensor 116 at home with a display unit in the same or a nearby room, or a hospital room. In some embodiments, there may be one or more processing and display units 118 and one or more of the units 118 can be located remotely from the person whose blood pressure is being monitored, such as a central monitoring station in a hospital. Additionally, a remote application could be for a person conducting at-home monitoring and their results are sent to a server at a health care facility for review by a health care professional associated with the person.

FIG. 3 illustrates an unrolled, example cuff with an altimeter 200. The example cuff 200 includes an inflation component 202, the altimeter 102, and an air port 204. The example cuff 200 can be any blood pressure measurement cuff known in the art. In embodiments, the example cuff 200 can also include securing components, such as Velcro, snaps, reusable adhesive, etc. Also, the example cuff 200 can be configured for use on an arm, wrist, leg etc. The example cuff 200 can be of different sizes, such as, varying infant, child, and adult sizes.

The example air port 204 can be configured to include one or more inputs or outputs, such as an air intake and an air outlet. Additionally, the example air port 204 can be configured to couple to one or more tubes for routing air into and out of the cuff's inflation component 202. In some embodiments, the cuff 200 does not have an air port 204 and one or more tubes are continuous with the internal bladder and extend outside the cuff to a measurement station.

The example cuff 202 has an x-y-plane that substantially conforms to one side of the cuff. In embodiments, the altimeter 102 is positioned on the cuff 202 such that the center of the altimeter 102 is at the same as the center of the air port 204 in the y-axis direction. In some embodiments, the altimeter 102 is positioned on the cuff 202 such that the center of the altimeter 102 is aligned with the center of the air port 204 in the x-axis direction. In other embodiments, the center of the altimeter 102 is positioned on the cuff 202 such that it is not in alignment in either the x- or y-directions with the center of the air port 204.

The example altimeter 102 can transmit a reading to a processing unit, not shown, via a wired or wireless connection. In embodiments, the altimeter 102 can be secured to the outside of the cuff 202. For instance, the altimeter 102 can be added to an existing cuff 202 as a retrofit. In some embodiments, the altimeter 102 can be secured to the interior of the cuff 202 in any of the locations previously described.

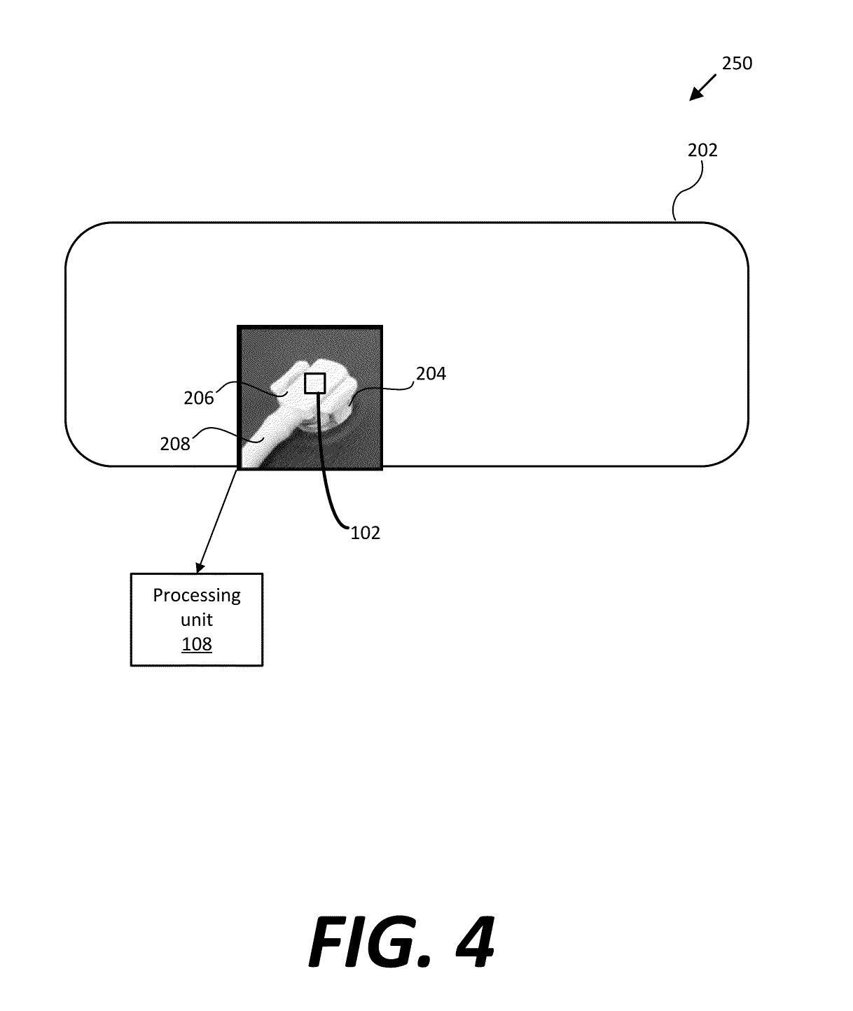

FIG. 4 illustrates another example 250 of the arrangement of the altimeter 102 in relation to the air port 204. The example embodiment 250 includes cuff 202, altimeter 102, air port 204, a connecting element 206 and a tubular element 208. The example tubular element 208 can be in fluid and/or electrical communication with a processing unit 108. In embodiments, the connecting element 206 can rotate when it is coupled to the air port 204.

Example configuration 200 shown in FIG. 3 optionally has an air port 204, whereas example configuration 250 requires an air port 204 configured to couple with connecting element 206. In embodiments, the connecting element 206 can be sized to couple to different sized air ports 204. In embodiments, the connecting element 206 and tubular element 208 are reused for different patients and a new cuff 202 is used for each patient. A commercial embodiment of disposable cuffs in connection with reusable connecting element 206 and tubular element 208 is the FlexiPort system manufactured by Welch Allyn (Skaneateles Falls, N.Y.).

In example 250, the altimeter 102 can be positioned inside the connecting element 206. Alternatively, the altimeter 102 can be positioned on the outer surface of the connecting element 206. In embodiments, the altimeter 102 communicates the one or more measurements via a wire that can pass through the tubular element to the processing unit 108. In other embodiments, the altimeter 102 is in communication with a processing unit 118 wirelessly.

FIG. 5 illustrates various locations for altimeters 102 and 104 on a human body 300. Showing locations on the human body is in no way intended to limit the application of the example systems to humans. The automated blood pressure measurement system can also be used in veterinary applications as well. In embodiments, the altimeters 102 and 104 can be positioned by the person or by a health care professional.

Generally, altimeter 102 can be placed wherever the blood pressure sensor 106 or 116 is applied on the body for measurement. As described above with reference to FIGS. 3 and 4, altimeter 102 can be secured to the cuff 202 or the connecting element 206. As shown in FIG. 5, altimeter 102 can be located in a person's upper arm, between the elbow and the hand, or on a finger. Additionally, altimeter 102 can be located on a person's thigh, calf, or ankle Altimeter 102 can be positioned on either side of a person's body. The locations depicted in FIG. 3, all on the front and right-hand side of body, are not intended to be limiting.

Generally, altimeter 104 can be positioned at the heart level of the person. Altimeter 104 can be affixed to a person or a person's clothing using any means known in the art, such as, for example, with medical tape, with a pin, or as a component in a patch. The location of altimeter 104 can vary depending on the orientation of the person's torso. For example, if the person is sitting or standing upright, altimeter 104 can be affixed anywhere on the person's torso provided it is at the same height from the ground as the person's heart. Alternatively, the altimeter 104 could be temporarily positioned at the heart elevation at the start of the blood pressure measurement and the system can use that altitude to correct the readings.

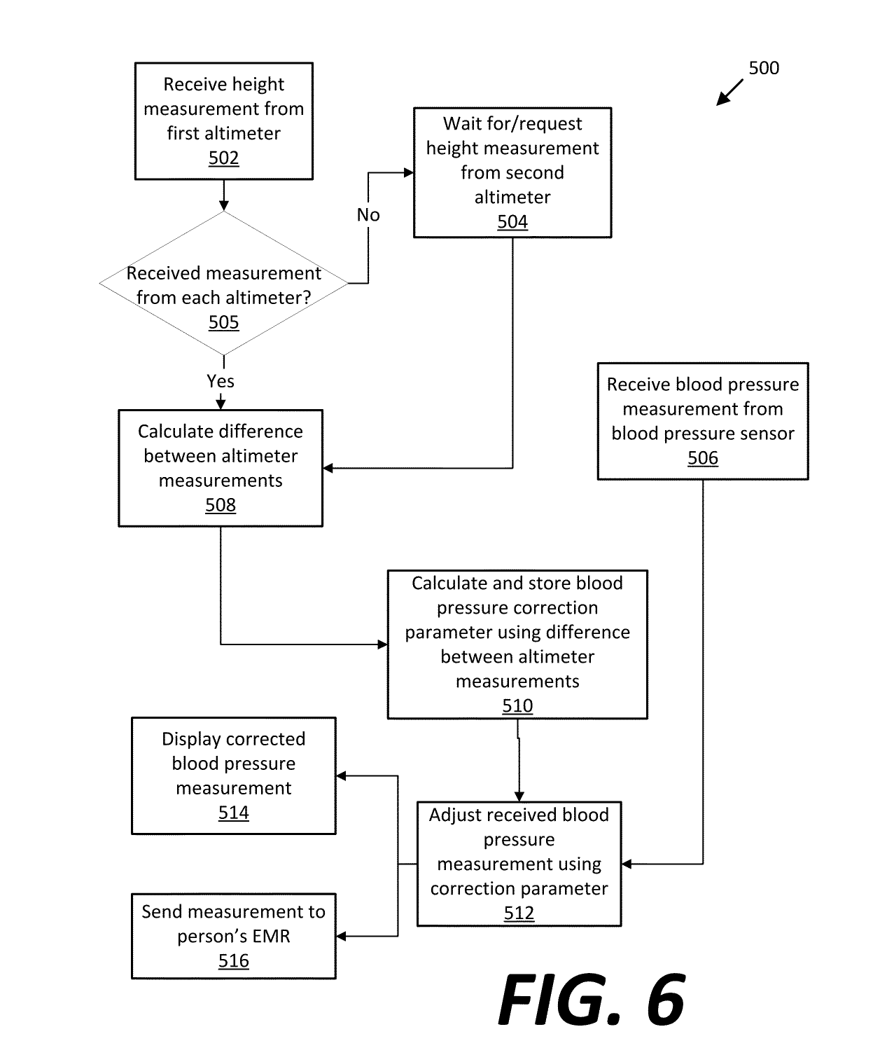

Each example method shown in FIGS. 6-10 can be preceded by one or more preparation steps that are not shown. For instance, in some embodiments, the altimeters and/or the blood pressure sensor can be configured to be in wired or wireless communication with one or more processing units. In embodiments, the altimeters can be calibrated before use in the example methods.

FIG. 6 is a flowchart illustrating a method 500 for using at least two altimeters in conjunction with a blood pressure sensor according to an example embodiment. In various embodiments, various steps in method 500 can be performed by one or more processing units. In some embodiments, the method can be initiated by a health care professional or by a person. “Person,” as used with reference to method 500, can be a patient in a medical facility or a person outside a medical facility who has initiated a measurement of their blood pressure.

Example method 500 includes receiving a height measurement from a first altimeter (step 502), determining whether a measurement has been received from each altimeter (step 505), waiting for or requesting a height measurement from a second altimeter (step 504), calculating the difference between altimeter measurements (step 508), calculating and storing a blood pressure correction parameter using the difference between altimeter measurements (step 510), receiving a blood pressure measurement from a blood pressure sensor (step 506), adjusting the received blood pressure measurement using a correction parameter (step 512), displaying a corrected blood pressure measurement (step 514) and sending the corrected measurement to a person's electronic medical record (step 516). Other embodiments may exclude some or all of these steps or add additional steps.

In this example, the use begins with a processing unit receiving a height measurement from a first altimeter (step 502). The processing unit can receive the height measurement from either the altimeter positioned at the blood pressure sensor or the altimeter positioned at the person's heart level. The processing unit can receive the height measurement via a wired or wireless communication protocol. In embodiments, the processing unit has already been paired to the two or more altimeters before step 502. In some embodiments, the processing unit can request a height measurement from one or more altimeters in a step before step 502, which can then initiate a height measurement by the one or more altimeters.

After the processing unit receives one height measurement, the processor determines whether it has received a measurement from each altimeter (step 505). If it has, then the processor calculates the difference between altimeter measurements (step 508). If the processor has received just one measurement, then the processor can wait for or request a height measurement from the other altimeter (step 504).

After the processing unit receives a height measurement from each altimeter, the processor calculates the difference between altimeter measurements (step 508). In some embodiments, the processor can calculate the absolute value of the difference between the two measurements. In other embodiments, the processor can calculate the height difference by subtracting the height measurement of the altimeter at the blood pressure sensor from the height measurement of the altimeter at the person's heart.

After the processor has determined the height difference between the altimeters, the next step in example method 500 is for the processor to calculate and store a blood pressure correction parameter (step 510). The blood pressure correction parameter uses as input the difference between altimeter measurements calculated in step 508. In embodiments, the correction parameter can be calculated using the following example formula(s):

Systolic_{corrected}=Systolic_{measured}+2(Altitude_{Measurement Site}−Altitude_{heart level})

Diastolic_{corrected}=Diastolic_{measured}+2(Altitude_{Measurement Site}−Altitude_{heart level})

MAP_{corrected}=MAP_{measured}+2(Altitude_{Measurement Site}−Altitude_{heart level})

At some point in example method 500, the processor receives a blood pressure measurement from the blood pressure sensor (step 506). As used herein, “blood pressure measurement” can mean a measurement comprising the systolic, diastolic and Mean Arterial Pressure blood pressure measurements. In embodiments, the processor can be coupled to a pressure sensing device that receives air via a tubing from a blood pressure cuff. In other embodiments, the blood pressure sensor measures and processes systolic and diastolic measurements and sends those measurements as a digital signal to the processor.

In embodiments, the example method 500 begins when the processor receives a measurement from the blood pressure sensor, and then the processor requests one or more measurements from the two or more altimeters. In some embodiments, the processor can send a request to the blood pressure sensor to send a measurement after the processor has received one or two height measurements from the two or more altimeters.

After the processor has received a height measurement at both the person's height and the blood pressure measurement site, as well as a blood pressure measurement, the processor next can adjust the received blood pressure measurement using the calculated correction parameter (step 512). Step 512 can include adjusting the received systolic and diastolic blood pressure measurements, as well as the mean arterial pressure. In embodiments, the processor can store the raw measurements and/or the adjusted elements in a storage device in electrical communication with the processor.

In embodiments, the processor can compare the times when the measurements were received. If one or more of the measurements were not received within a given tolerance range, for example, 0.1 microsecond, 0.2 microsecond, 0.5 microsecond, 1 microsecond, 2 microseconds, or 3 microseconds, the processor can request new measurements. In some instances, when an unacceptably long period of time elapses between measurements, the person can move an appendage supporting the blood pressure sensor or altered their posture. Thus, in order for the correction parameter to accurately compensate for any difference in height in this embodiment, the height measurements and the blood pressure measurement must be performed within a given tolerance range.

When the processor has adjusted the blood pressure measurement, the corrected measurement can be displayed (step 514). In embodiments, the display unit can be located near the person whose blood pressure has been measured. In embodiments, the corrected measurement can be displayed as part of a monitoring area where more than one person's vital signs are being monitored simultaneously.

Alternatively, or in addition to step 514, the processor can send the corrected measurement to a person's electronic medical record (EMR) for storage and potential future use or viewing (step 516). In embodiments, there may be one or more alarm limits set in the patient's EMR for either the systolic pressure, diastolic pressure, or both. These alarm limits can be configured to trigger a notification of a health care professional associated with the person when one of the limits is exceeded.

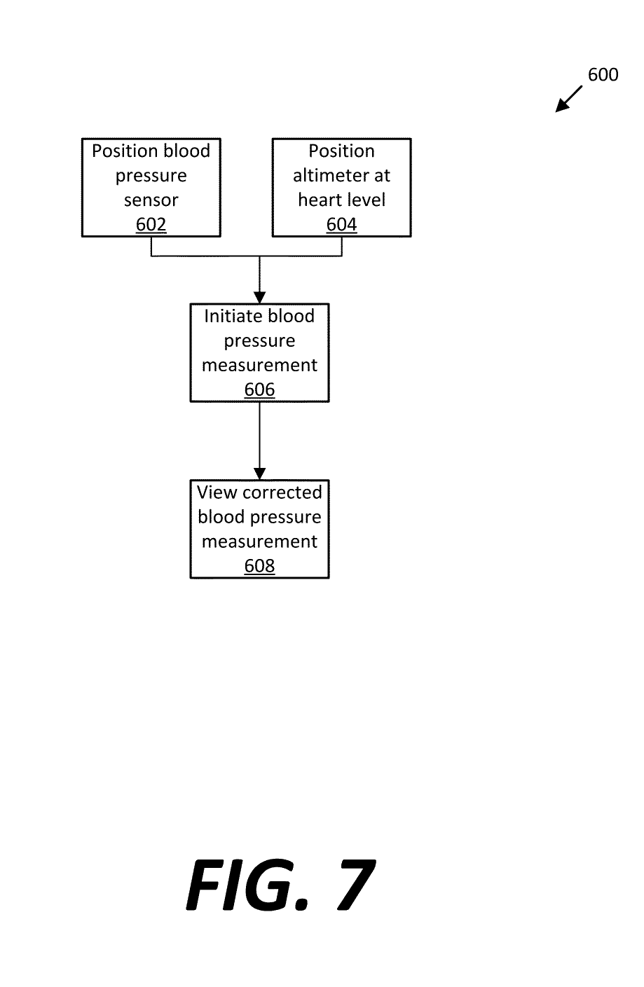

FIG. 7 illustrates a flowchart of an example method of using an altimeter-blood pressure sensor system 600. The steps of example method 600 can be performed by one or more health care professionals, a person who is monitoring their blood pressure, or one or more people helping or associated with that person. Accordingly, the following discussion of example method 600 leaves open which entity can perform each step.

Example method 600 includes positioning a blood pressure sensor (step 602), positioning an altimeter at heart level (step 604), initiating a blood pressure measurement (step 606) and viewing the corrected blood pressure measurement (step 608). Other embodiments may exclude some or all of these steps or add additional steps.

In example method 600, the use can begin by positioning a blood pressure sensor (step 602) somewhere on the person's body. A first altimeter is supported by the blood pressure sensor or is integral with the blood pressure sensor. Locations of where the blood pressure sensor can be placed are described in more detail above at least with reference to FIG. 5. Configurations of the blood pressure sensor and where the altimeter is positioned relative to the sensor are described in more detail above at least with reference to FIGS. 1-4. In embodiments, prior to step 602 the altimeter may have been paired to communicate with a processing unit.

In example method 600, the use can also begin by positioning a second altimeter at the person's heart level (step 604). Possible locations of the second altimeter relative to the heart are described in more detail above with reference to FIG. 5.

Once the blood pressure sensor and second altimeter are positioned, the blood pressure measurement is initiated (step 606). In embodiments, the blood pressure measurement can be accomplished automatically by a blood pressure measurement device or with the assistance of a health care professional.

After the blood pressure measurement, the corrected blood pressure measurement can be viewed (step 608). An example determination of a corrected blood pressure measurement is shown and described in more detail above with reference to FIG. 6. As described above, the corrected measurement can be viewed in the immediate vicinity of the person, somewhere in the same facility as the person, or in a separate location.

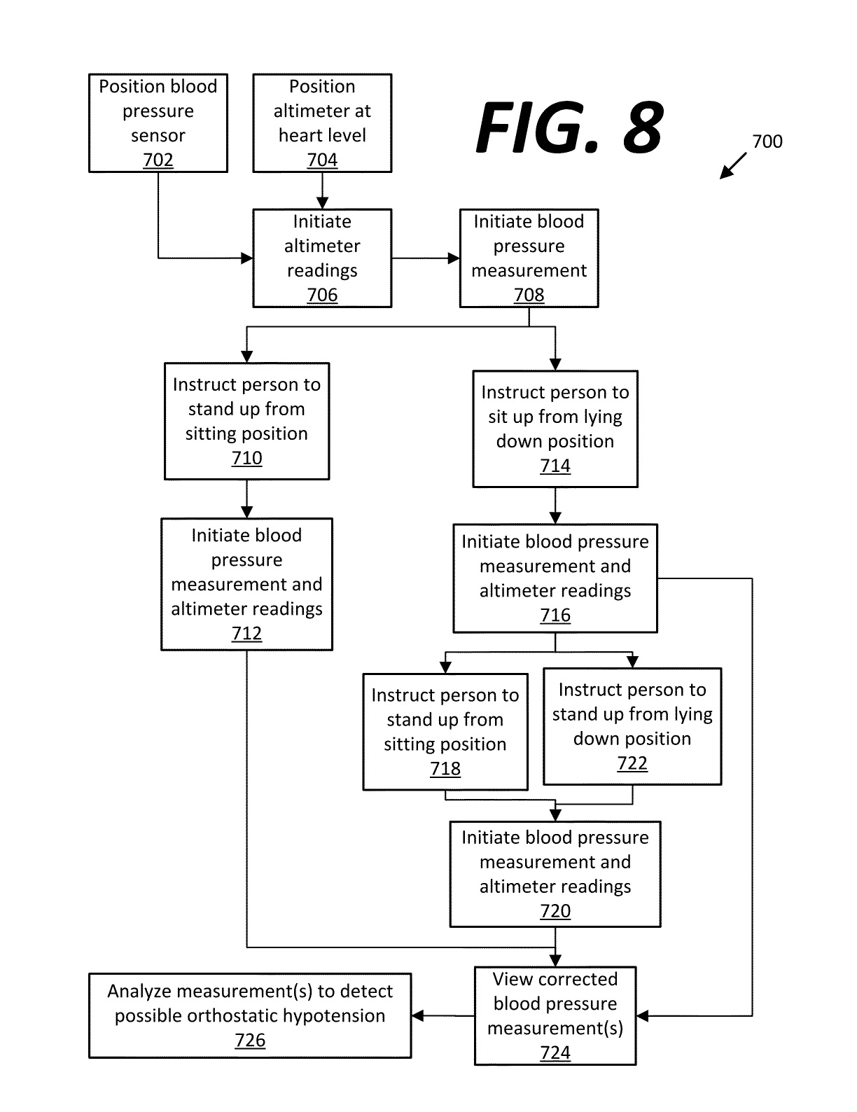

FIG. 8 illustrates an example method of conducting an orthostatic blood pressure test 700. Similar to example methods 500 and 600, example method 700 can be conducted in a health care facility, a monitored-care facility, or in a home-type setting. In some situations, a person's blood pressure is measured over a period of time as that person changes orientation or position. Thereby a person can be screened for orthostatic hypotension.

The example method 700 includes positioning a blood pressure sensor (step 702), positioning an altimeter at heart level (step 704), initiating one or more altimeter readings (step 706), initiating a blood pressure measurement (step 708), instructing a person to stand up from a sitting position (step 710), instructing a person to sit up from a lying down position (step 714), instructing a person to stand up from a sitting position (step 718), instructing a person to stand up from a lying down position (step 722), viewing one or more corrected blood pressure measurements (step 724), initiate blood pressure measurement and altimeter readings (steps 712, 716 and 720), and analyzing the one or more measurements to detect possible orthostatic hypotension (step 726).

Example use 700 can begin by positioning the blood pressure sensor on the person's body (step 702). Locations of where the blood pressure sensor can be placed are described in more detail above at least with reference to FIG. 5. A first altimeter is supported by the blood pressure sensor or is integral with the blood pressure sensor. Configurations of the blood pressure sensor and where the altimeter is positioned relative to the sensor are described in more detail above at least with reference to FIGS. 1-4.

Example use can also begin by positioning a second altimeter at the person's heart level (step 704). Possible locations of the second altimeter relative to the heart are described in more detail above with reference to FIG. 5. In embodiments, the altimeter is positioned such that it will accurately reflect the height of the heart throughout the person's changing positions in steps 710, 712 and 714.

After the blood pressure sensor and altimeters are positioned, the altimeter readings are initiated (step 706). In embodiments, the blood pressure measurement (step 708) is initiated simultaneously or shortly after the altimeter readings are initiated. In embodiments, the measurements can be initiated from a control unit that is in communication with the altimeters and blood pressure sensor. The measurements in steps 706 and 708 can provide a baseline blood pressure that can be compared against the results of steps 712, 716 and/or 720.

After the baseline readings have been measured in steps 706 and 708, the health care professional can next instruct the person to stand up from a sitting position (step 710) or sit up from a lying down position (step 714). When the person has stood up (step 710) or sat up (step 714), another blood pressure measurement and altimeter readings are initiated (step 712 or 716). After one or more processing units receive the measurements in steps 712, 716, or 720, the one or more processing units can perform a blood pressure correction similar to example method 500 shown in FIG. 6.

After the measurements have been recorded in step 712, the health care professional can ask the person to sit down and then record another round of blood pressure and altimeter measurements (not shown). Alternatively, after initiating the blood pressure and altimeter measurements (step 712), a health care professional can view the corrected blood pressure measurements on a display (step 724). In embodiments, the health care professional can repeat one or more of steps 710-720, not shown in FIG. 8.

After the blood pressure and altimeter measurements have been recorded (step 716), the health care professional can view the results (step 724) or instruct the person to stand up from a sitting position (step 718). In some embodiments, the person can be instructed to lie back down after sitting up, which can optionally be followed by another round of measurements, not shown.

In embodiments, the health care professional can next instruct the person to stand up from a sitting position (step 718) or stand up from a lying down position (not shown). When the person has stood up, a blood pressure measurement and altimeter readings can be initiated (step 720).

In embodiments, the system can be configured to provide continuous or rapid (e.g., every 0.1 second) measurements during the person's motion, for example, sitting up, standing up, sitting down, lying down, etc. Those results can be presented as a graph or chart in step 724.

After the one or more blood pressure measurements have been corrected and viewed (step 724), the measurements can be analyzed to detect possible orthostatic hypotension (step 726). In embodiments, the results are analyzed by one of the health care professionals participating in one or more of the steps of example method 700. In other embodiments, the results can be analyzed by a health care professional who did not participate in any of the steps of example method 700. In this case, the altitude samples can be time-aligned with the segments of the blood pressure determination cycle. For example, the specific time segment when the systolic pressure was recorded during the blood pressure measurement could be corrected using the altitude measurements aligned with that segment in time.

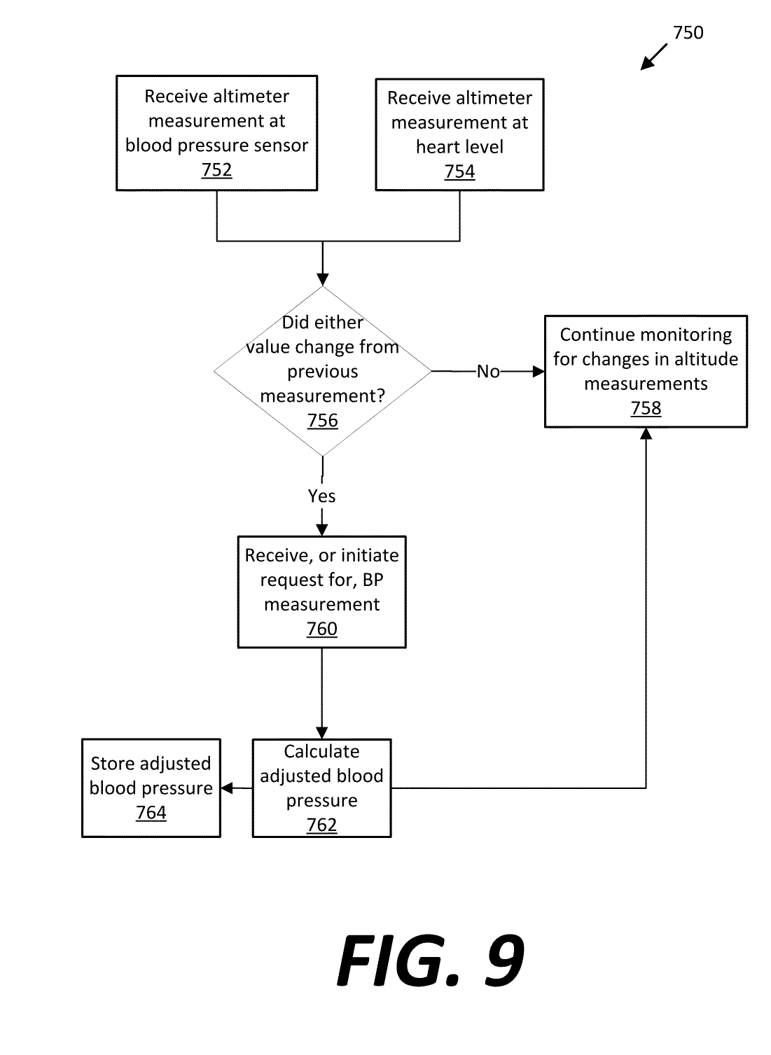

FIG. 9 illustrates an example method of a processing unit monitoring two or more altimeters 750. The example method 750 includes receiving an altimeter measurement from the sensor positioned at the blood pressure sensor (step 752), receiving an altimeter measurement from an altimeter positioned at the patient's heart level (step 754), evaluating whether either value changed from a previous measurement (step 756), continuing to monitor for changes in altitude measurements (step 758), receiving or initiating a request for a blood pressure measurement (step 760), calculating an adjusted blood pressure (step 762) and storing the adjusted blood pressure (step 764). Example method 750 can be implemented by one or more processors. Example method 750 can be conducted during, for example, an orthostatic hypotension test. Other embodiments can include more steps or exclude steps.

Example method 750 can begin when one of the processing units receives a height measurement from the altimeter positioned at the blood pressure sensor (step 752) or from the altimeter positioned at the person's heart level (step 754). In some embodiments, either or both altimeters can be configured to send a height measurement on a periodic basis to a paired processing unit, such as every 0.05 second, every 0.1 second, every 0.25 second, every 0.5 second and every 1 second. In embodiments, the processor can store the received height measurement along with other data, such as the time and date the measurement was sent.

After the processing unit receives at least one height measurement, the processing unit can determine whether the received measurement changed from a previously-received measurement (step 756). In embodiments, the processor accesses a local or cloud-based storage device to search for prior measurements received from the altimeter. In embodiments, step 756 can include subtracting the previous measurement, which can be zero if there was no previously-received measurement, from the current measurement. In embodiments, the evaluation in step 756 can include a minimum threshold for a “yes”, such as, at least 0.1 inch, 0.25 inch, 0.75 inch or 1 inch.

If the result of the determination in step 756 is “no,” then the processing unit continues to monitor for changes in altitude measurements (step 758). Continuing to monitor could include entering a sleep state until the processing unit receives a wake-up signal or a new measurement from one of the altimeters in the system.

If the result of the determination in step 756 is “yes”, then the processing unit can receive or initiate a request for a blood pressure measurement from the blood pressure sensor. In embodiments, initiating a request for a blood pressure measurement can be a message to a health care professional, such as a text message, email, or display on a monitor. In embodiments, initiating a request for a blood pressure measurement can be an electrical signal to a paired blood pressure sensor that is capable of automatically initiating and conducting a blood pressure measurement.

After receiving the blood pressure measurement, the processing unit next calculates an adjusted blood pressure (step 762). In some embodiments, the adjusted blood pressure is calculated in accordance with example method 500 shown in FIG. 6.

The adjusted blood pressure can then be stored in a local or cloud storage medium (step 764). In embodiments, other parameters can be stored in addition to the adjusted blood pressure, such as, time, date, heart rate, temperature, saturated oxygen, etc. In some embodiments, the adjusted blood pressure can be stored in a patient's electronic medical record.

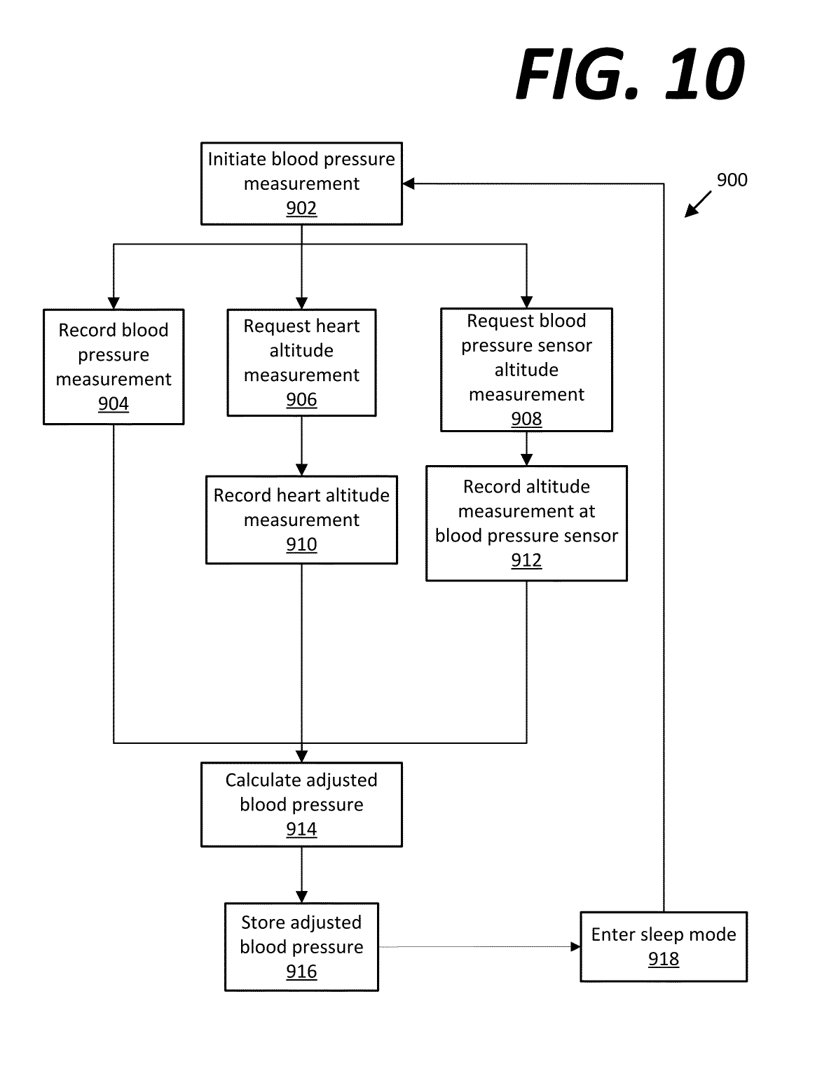

FIG. 10 illustrates an example method of a processing unit's interaction with a mobile blood pressure sensor 900. Example method 900 includes initiating a blood pressure measurement (step 902), recording a blood pressure measurement (step 904), requesting an altitude measurement at the heart level (step 906), requesting an altitude measurement at the blood pressure sensor (step 908), recording an altitude measurement from the altimeter at heart level (step 910), recording the altitude measurement at the blood pressure sensor (step 912), calculating an adjusted blood pressure (step 914), storing the adjusted blood pressure (step 916) and entering a sleep mode (step 918). Example method 900 can be implemented by one or more processors. Other embodiments can include more steps or exclude steps.

The blood pressure sensor used in conjunction with example method 900 is worn on the person and can be worn outside of a hospital or care facility. In some embodiments, the worn sensor can include a blood pressure sensor component, processing and storage components, a wireless communication component, and an altimeter. Additionally, a second altimeter is positioned or can be positioned at the person's heart level.

Example method 900 begins by initiating a blood pressure measurement (step 902). In embodiments, the processor can be configured to initiate a blood pressure measurement at predetermined intervals, such as every 30 minutes, every hour, every 2 hours, every 4 hours, or every 8 hours. In some embodiments, the processing unit can receive a request to initiate a blood pressure measurement wirelessly, for example, over a Wi-Fi connection from a health care professional associated with the person.

After initiating the blood pressure measurement, the processing unit records the blood pressure measurement (step 904). Simultaneously or subsequently, the processing unit can request height measurements at the heart level (step 906) and at the blood pressure sensor (step 908).

After requesting and receiving the height measurements from the altimeters, the processing unit records the altimeter's height measurement at the heart level (step 910) and at the blood pressure sensor (step 912). The blood pressure and height measurements can be stored locally and can also include additional data, such as, the date, time or GPS data when the blood pressure measurement was conducted.

After the processing unit has received the blood pressure measurement and the two height measurements, the processor calculates the adjusted blood pressure (step 914). The adjusted blood pressure can be calculated using a method similar to that shown and described with reference to FIG. 6.

After the adjusted blood pressure is calculated, the processor can store the result (step 916). In some embodiments, the adjusted blood pressure can be immediately transmitted to a remote processing unit, such as a server at a health care facility or to a computing unit of the person. In some embodiments, the processing unit enters a sleep mode (step 918) until prompted to initiate another blood pressure measurement.

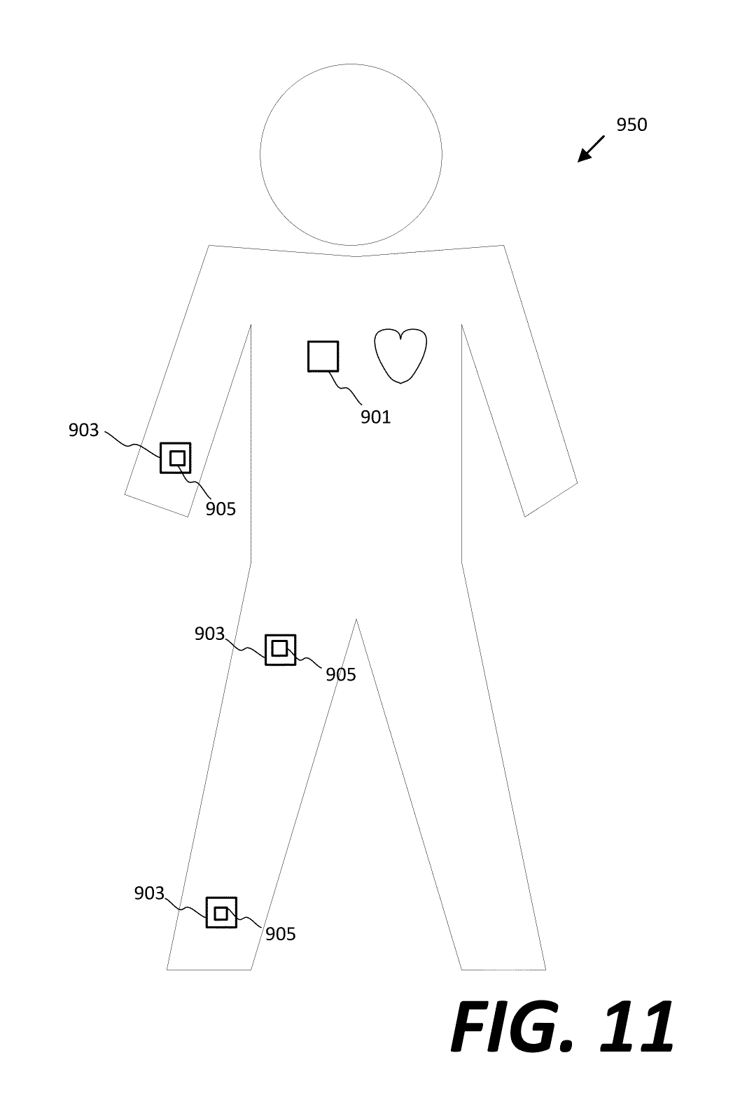

FIG. 11 illustrates various locations for the wearable blood pressure sensor including altimeter on a human body 950. The example arrangements show the possible position of a blood pressure sensor 903 with an integral altimeter 905 and an altimeter 901 positioned at the person's heart level. Showing locations on the human body is in no way intended to limit the application of the example systems to humans. The automated blood pressure measurement system can also be used in veterinary applications as well. The example arrangement 950 can be used in conjunction with example method 900 shown in FIG. 10.

In embodiments, the sensor 903 can be secured to the person's body using, for example, a fixed-length band or a Velcro strap. As depicted in FIG. 11, the sensor 903 can be positioned at a person's wrist, thigh, or lower leg. In embodiments, the sensor 903 can be continuously worn or worn during mild, moderate or heavy physical activity. Altimeter 901 can be affixed to a person's shirt or directly to the person's skin underneath their clothing. In some embodiments, altimeter 901 is only positioned at the heart level during blood pressure measurements. For example, the sensor 903 can emit a vibration or audible signal that it is about to begin a measurement, which can cue the person to affix altimeter 901 to be positioned at heart level.

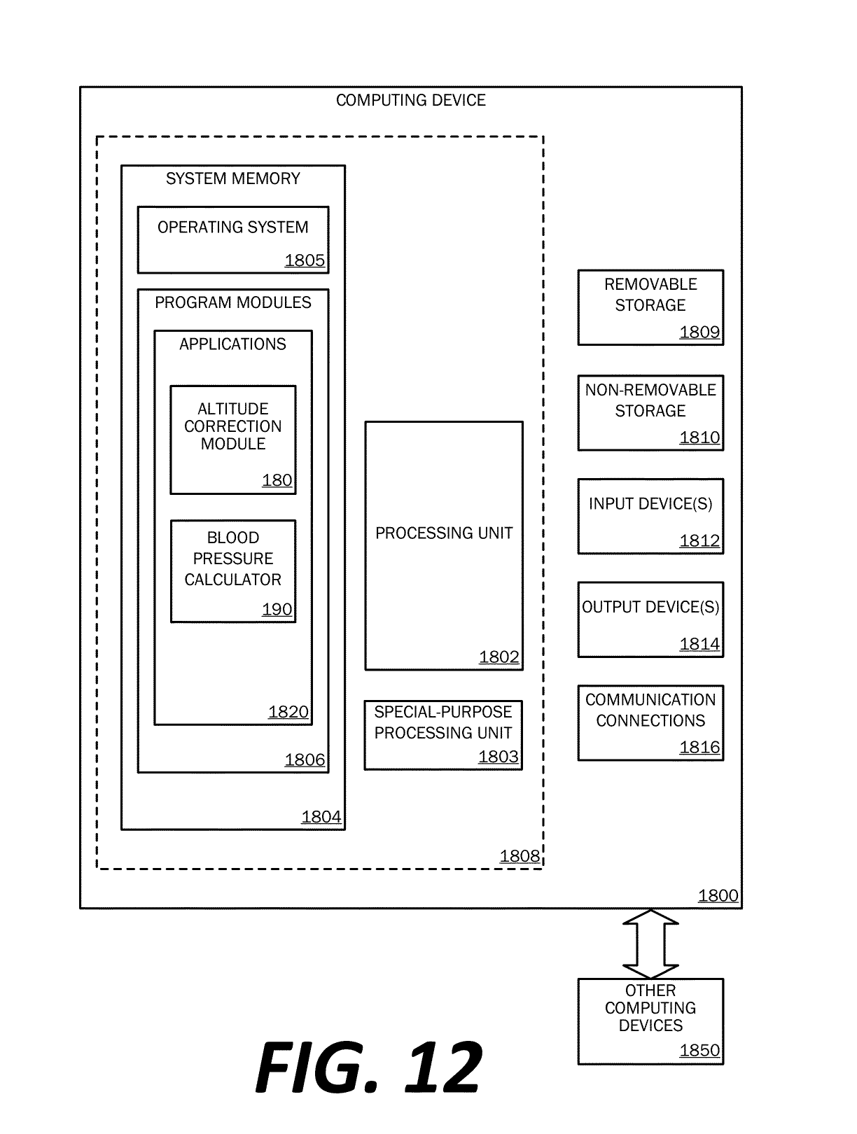

FIG. 12 is a block diagram illustrating physical components (i.e., hardware) of a computing device 1800 with which embodiments of the disclosure may be practiced. The computing device components described below may be suitable to act as the computing devices described above, such as processing unit 108 of FIG. 1. In a basic configuration, the computing device 1800 may include at least one processing unit 1802 and a system memory 1804. Depending on the configuration and type of computing device, the system memory 1804 may comprise, but is not limited to, volatile storage (e.g., random access memory), non-volatile storage (e.g., read-only memory), flash memory, or any combination of such memories. The system memory 1804 may include an operating system 1805 and one or more program modules 1806 suitable for running software applications 1820 such as the altitude correction module 180 and blood pressure calculator 190. The operating system 1805, for example, may be suitable for controlling the operation of the computing device 1800. Furthermore, embodiments of the disclosure may be practiced in conjunction with a graphics library, other operating systems, or any other application program and is not limited to any particular application or system. This basic configuration is illustrated in FIG. 12 by those components within a dashed line 1808. The computing device 1800 may have additional features or functionality. For example, the computing device 1800 may also include additional data storage devices (removable and/or non-removable) such as, for example, magnetic disks, optical disks, or tape. Such additional storage is illustrated in FIG. 12 by a removable storage device 1809 and a non-removable storage device 1810.

As stated above, a number of program modules and data files may be stored in the system memory 1804. While executing on the processing unit 1802, the program modules 1806 (e.g., altitude correction module 180 and blood pressure calculator 190) may perform processes including, but not limited to, calculating adjustment parameters and calculating corrected blood pressure measurements, as described herein. Other program modules that may be used in accordance with embodiments of the present disclosure, and in particular to generate screen content, may include electronic mail and contacts applications, word processing applications, spreadsheet applications, database applications, slide presentation applications, drawing or computer-aided application programs, etc.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. For example, embodiments of the disclosure may be practiced via a system-on-a-chip (SOC) where each or many of the components illustrated in FIG. 12 may be integrated onto a single integrated circuit. Such an SOC device may include one or more processing units, graphics units, communications units, system virtualization units and various application functionality all of which are integrated (or “burned”) onto the chip substrate as a single integrated circuit. When operating via an SOC, the functionality, described herein, with respect to the altitude correction module 180 and blood pressure calculator 190 may be operated via application-specific logic integrated with other components of the computing device 1800 on the single integrated circuit (chip). Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general purpose computer or in any other circuits or systems.

The computing device 1800 may also have one or more input device(s) 1812 such as a keyboard, a mouse, a pen, a sound or voice input device, a touch or swipe input device, etc. The output device(s) 1814 such as a display, speakers, a printer, etc. may also be included. The aforementioned devices are examples and others may be used. The computing device 1800 may include one or more communication connections 1816 allowing communications with other computing devices 1818. Examples of suitable communication connections 1816 include, but are not limited to, RF transmitter, receiver, and/or transceiver circuitry; universal serial bus (USB), parallel, and/or serial ports.

The term computer readable media as used herein may include non-transitory computer storage media. Computer storage media may include volatile and nonvolatile, removable and non-removable media implemented in any method or technology for storage of information, such as computer readable instructions, data structures, or program modules. The system memory 1804, the removable storage device 1809, and the non-removable storage device 1810 are all computer storage media examples (i.e., memory storage.) Computer storage media may include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information and which can be accessed by the computing device 1800. Any such computer storage media may be part of the computing device 1800. Computer storage media does not include a carrier wave or other propagated or modulated data signal.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

Embodiments of the present disclosure may be utilized in various distributed computing environments where tasks are performed by remote processing devices that are linked through a communications network in a distributed computing environment.

The flow diagrams depicted herein are just examples. There may be many variations to these diagrams or the steps (or operations) described therein without departing from the spirit of the disclosure. For instance, the steps may be performed in a differing order, or steps may be added, deleted or modified.

While embodiments have been described, it will be understood that those skilled in the art, both now and in the future, may make various improvements and enhancements can be made.

What is claimed is:

1. A non-invasive blood pressure measurement system, comprising:
a processing unit;
a non-invasive blood pressure sensor configured to transmit a blood pressure signal to the processing unit;
a first altitude sensor configured to generate a first height measurement and transmit the first height measurement to the processing unit; and
a second altitude sensor configured to generate a second height measurement and transmit the first height measurement to the processing unit;
wherein the processing unit is configured to generate an adjusted blood pressure measurement using the first height measurement, the second height measurement and the blood pressure signal.

2. The non-invasive blood pressure measurement system of claim 1, wherein the non-invasive blood pressure sensor is a cuff including a pressure interface opening, and wherein the first altitude sensor is positioned at the cuff.

3. The non-invasive blood pressure measurement system of claim 2, wherein the processing unit is configured to calculate the adjusted blood pressure measurement after receiving both the first and the second height measurements.

4. The non-invasive blood pressure measurement system of claim 1, further comprising a display module, the display including the calculated systolic and diastolic blood pressure.

5. The non-invasive blood pressure measurement system of claim 1, wherein one or both of the first and the second altitude sensors are configured to transmit the height measurement to the processing unit wirelessly.

6. The non-invasive blood pressure measurement system of claim 5, wherein the blood pressure signal comprises the unadjusted systolic and diastolic blood pressure.

7. The non-invasive blood pressure measurement system of claim 6, wherein the blood pressure sensor is configured to transmit the blood pressure signal to the processing unit wirelessly.

8. The non-invasive blood pressure measurement system of claim 1, wherein the second altitude sensor is positioned at heart level of a patient.

9. A method for determining the blood pressure of a patient, comprising:
receiving a blood pressure measurement from a blood pressure sensor;
receiving a first height measurement from a first altimeter;
receiving a first height measurement from a second altimeter;
adjusting the blood pressure measurement using the first height measurement from the first altimeter and the first height measurement from the second altimeter;
producing an adjusted blood pressure measurement comprising the systolic pressure, diastolic pressure and mean arterial pressure; and
displaying the adjusted blood pressure measurement.

10. The method of claim 9, further comprising:
positioning the first altimeter at the blood pressure sensor; and
positioning the second altimeter at a heart of the patient.

11. The method of claim 9, wherein the patient is a person.

12. The method of claim 9, wherein the patient is an animal.

13. The method of claim 9, wherein the blood pressure measurement is received from a pressure transducer.

14. The method of claim 9, wherein each of the blood pressure measurement, the first height measurement and the second height measurement are received wirelessly.

15. The method of claim 9, further comprising:
receiving a second height measurement from the first altimeter; and
initiating a second blood pressure measurement.

16. The method of claim 15, further comprising:
receiving a second height measurement from the second altimeter; and
adjusting the second blood pressure measurement using the second height measurement from the first altimeter and the second height measurement from the second altimeter, producing a second adjusted blood pressure measurement comprising the systolic and diastolic pressure.

17. The method of claim 16, wherein the adjusted blood pressure measurement and the second adjusted blood pressure measurement are used to diagnose orthostatic hypotension.

18. The method of claim 16, wherein the blood pressure sensor is a cuff including a pressure interface opening, and wherein the first altimeter is affixed to the cuff.

19. The method of claim 9, wherein the second altimeter is positioned at the heart level of the patient.

20. A tangible, non-transitory computer readable medium containing computer executable instructions which, when executed by a computer, perform a method for determining a corrected blood pressure measurement, the method comprising the steps of:
receiving a blood pressure measurement from a blood pressure sensor;
receiving a first height measurement from a first altimeter;
receiving a first height measurement from a second altimeter;
adjusting the blood pressure measurement using the first height measurement from the first altimeter and the first height measurement from the second altimeter;
producing an adjusted blood pressure measurement comprising the systolic and diastolic pressure; and
displaying the adjusted blood pressure measurement.
